(19) United States
(12) Patent Application Publication
(10) Pub. No.: US 2011/0311633 A1
(43) Pub. Date: Dec. 22, 2011

(54) Combination of a Silicon Containing Component and a Hormone

(76) Inventors: Leigh Trevor Canham, Worcestershire (GB); Jonathan Joseph Powell, Cambridge (GB); Ravin Jugdaohsingh, London (GB)

(21) Appl. No.: 11/885,669

(22) PCT Filed: Mar. 3, 2006
(86) PCT No.: PCT/GB2006/000784

§ 371 Date: Oct. 3, 2008

(30) Foreign Application Priority Data
Mar. 5, 2005 (GB) 0504657.8

(51) Int. Cl. A61K 33/00; A61P 19/10; A61K 9/14

(52) U.S. Cl. 424/489; 424/600

(57) ABSTRACT

The invention relates to the use of a silicon-containing material to reduce the amount of hormone required in a method of hormone treatment to achieve a desired response, particularly in hormone replacement therapy (HRT) and especially in the maintenance of post-menopausal bone health and the management or treatment of osteoporosis, and to pharmaceutical compositions for use in such a method.

### FIELD OF THE INVENTION

The present invention relates to improvements in methods of hormone-related therapies, particularly oestrogen-based treatments. In particular, the invention relates to the use of a silicon containing material to reduce the amount of oestrogen required in a method of oestrogen treatment to achieve a desired response, particularly in hormone replacement therapy (HRT) and especially in the maintenance of post-menopausal bone health and the management or treatment of osteoporosis, and to pharmaceutical compositions for use in such a method.

### BACKGROUND TO THE INVENTION

Compounds having oestrogenic activity have been proposed for the treatment or prevention of a wide variety of conditions related to oestrogen functioning. Oestrogen replacement therapy, for example, has been reported to be effective in inhibiting or preventing diseases or conditions associated with reduction in endogenous oestrogen such as post-menopausal symptoms, including mood swings, depression, menopausal flash, insomnia, itching, osteoporosis and cardiovascular disease. Oestrogen is also widely used as a contraceptive agent.

Despite the therapeutic utility of oestrogen, the side effects associated with long term administration have limited its use. Long term oestrogen therapy has been implicated in an increased risk of cancer, particularly breast, uterine and endometrial cancers, for example, and this has led to considerable interest in the development of alternative therapies which have the desired beneficial effects without the undesirable side effects associated with long term, high dose oestrogen treatments.

Osteoporosis is a bone disorder which is associated with low bone mass and degradation of bone tissue, leading to increased bone fragility and risk of fracture. It is especially prevalent in post-menopausal women. To date, the risk of osteoporosia has been one of the main reasons for the prophylactic administration of oestrogen in post-menopausal women. In addition, administration of oestrogen has been one of the main treatments and maintenance therapies for post-menopausal osteoporosis to be suggested in the literature, as this has been found to slow bone loss.

In particular, oestrogen replacement therapy is a recognised treatment for osteoporosis in post-menopausal women. Concerns about the side effect profile have led to moves to use lower doses of oestrogen for the treatment of post-menopausal related problems although this remains controversial as it has been suggested that the beneficial effects to bone may be lost (Runowicz, Health News, 9(11), 12-13, 2003 and Crandall, J. Womens Health, 12(8), 723-47, 2003).

A variety of other compounds have been used or proposed as antiosteoporosis agents including oestrogen receptor modulators (as described, for example, in U.S. Pat. No. 6,750,213), bisphosphonates and other bone-active phosphonates including combinations with oestrogen (see for example, U.S. Pat. No. 6,329,354) and isoflavones in combination with oestrogen as described in U.S. Pat. No. 6,326,666. Osteoblasts, which are present in bone and which are responsive to oestrogen are also sensitive to a range of other hormones, such as progesterone and also thyroid hormones, such as T3. These hormones may also be useful in therapy, either alone or in combination with oestrogen.

Silicon-containing products for use in the prevention, treatment or delaying the onset of bone disease such as post-menopausal osteoporosis are described in U.S. Pat. No. 4,870,191. Dietary silicon (as the soluble orthosilicate anion) has been implicated as being important in bone formation (Reffitt et. al. Bone. 2003: 32; 127-135) and recent studies have shown that dietary silicon intake is positively associated with bone mineral density in men and premenopausal women (Journal of Bone and Mineral Research, 19 (2), 297-307, 2004). Studies in mature ovariectomised rats have suggested that soluble organo-silicon supplementation can help to prevent bone loss and increase bone formation rates (Calcif. Tissue Int. 53, 174-179 (1993)) and in a commentary on this study it is postulated that although the supplemental silicon does not greatly reduce bone loss, silicon supplementation in conjunction with hormone replacement could be considered to help prevent osteoporosis (http://www.prolithic.com/hpages/ref_docs/orthosil.html). There is however no suggestion that administering silicon in conjunction with the hormone would lead to unexpectedly advantageous or synergistic results.

It has previously been reported in the literature that the absorption and tissue metabolism of silicon is influenced by hormonal and endocrine functions (see Charnot et. al., Lyon Medicine 13, 85 (1971)) but there has been no suggestion, conversely, that silicon has any effect on hormone function. There remains a continuing interest in the development of improved hormone treatments and in particular in developing methods with reduced side effects compared to treatments known in the art. Alternative osteoporosis therapies are particularly sought.

### SUMMARY OF THE INVENTION

The present inventors have found that if an oestrogen is administered together with a silicon containing component in a method of oestrogen treatment, the amount of oestrogen required to achieve the desired effect is reduced compared to the amount which would otherwise be required in the absence of the silicon containing component.

By means of the invention, improved oestrogen treatments compared to known treatments are provided wherein the oestrogen can be administered in a lower daily dose than would otherwise be necessary to achieve the desired clinical effect, thereby reducing the risk from side effects associated with the long term use of high doses of oestrogens.

Administration of an oestrogen together with a silicon containing component in this manner will therefore be particularly effective in treating or preventing osteoporosis in humans or other animals. Not only will administration of the oestrogen with the silicon containing component give rise to much greater improvements in bone health than are observed with either oestrogen or silicon containing component alone but the effects will also be greater than the sum of the individual component effects.

The present inventors have also found that increases in activity of osteoblast cells similar to those produced by oestrogen in combination with a silicon containing component can also be obtained by administering other hormones in the presence of a silicon-containing component. In particular, both the sex hormone progesterone and the thyroid hormone triiodothyronine, T3, have been found to show this effect. Moreover, this effect has been found to be observed not only in pre-osteoblast cells but also in other cells of osteoblast lineage such as the cells of an osteosarcoma cell line. Administration together with a silicon containing component therefore affords the possibility of lowering the dose necessary to achieve the desired clinical effect for these hormones also in the treatment or prevention of conditions for which enhancing the level of the hormone is indicated to be beneficial.

The use of porous silicon as the silicon containing component is particularly advantageous. Porous silicon erodes to form silicic acid, particularly orthosilicic acid, in the gastrointestinal tract or when implanted and this silicic acid can be taken up by cells, for example bone cells. Porous silicon may also advantageously act as a permeation enhancer for dermal delivery of oestrogen. Moreover, the properties of porous silicon render it useful as a vehicle for delivering the oestrogen to the subject; formulations in which the oestrogen is at least partly located in the pores of the porous silicon provide protection against degradation of the oestrogen in the gastrointestinal tract and also afford the possibility of controlled release of the oestrogen disposed in the pores of the porous silicon as a result of corrosion or dissolution of the resorbable silicon.

The administration of porous silicon together with a hormone such as an oestrogen has not previously been described in the literature.

According to a first aspect, therefore, the present invention provides the use of a silicon containing component to reduce the amount of hormone required to be administered per day to a human or animal subject in order to achieve the desired therapeutic effect in a method of hormone treatment.

According to another aspect, the invention also provides the use of a hormone and a silicon containing component in the manufacture of a medicament for the treatment or prevention of a condition in a human or other animal subject in which enhancing the level of hormone is indicated to be beneficial, wherein the amount of hormone administered per day is lower than the normal effective dose of hormone per day in the treatment in the absence of the silicon containing component.

Also provided is a method for treating or preventing a condition in which enhancing the level of hormone is indicated to be beneficial in a human or other animal subject comprising administering to the subject a silicon containing component and administering to the subject a hormone in an amount per day which is lower than the normal effective dose of hormone per day in the treatment in the absence of the silicon containing component.

In a further aspect, the invention provides a pharmaceutical composition for co-administration according to the above aspects comprising a silicon containing component and a hormone, wherein the hormone is present in an amount which is lower than the normal effective dose of hormone per day in the treatment in the absence of the silicon containing component.

According to another aspect, the invention provides the use of porous silicon and a hormone in the manufacture of a medicament for the treatment or prevention of a condition in which enhancing the level of hormone is indicated to be beneficial in a human or other animal subject. Also provided is a method of treating or preventing a condition in which enhancing the level of hormone is indicated to be beneficial in a human or other animal subject comprising administering to the subject porous silicon and a hormone.

In a further aspect, the invention provides a pharmaceutical composition comprising porous silicon and a hormone and the use of such a composition in therapy in a human or other animal subject.

### DETAILED DESCRIPTION OF THE INVENTION

A hormone for use according to the invention may be a naturally occurring or synthetic hormone such as are well known in the art.

Suitable hormones for use according to the invention include, but are not limited to, hormones which have oestrogen pathway interacting activity such as humic substances (naturally occurring polysaccharides) or bone influencing activity, for example androgens, such as testosterone, parathyroid hormone, parathyroid hormone-relating protein, calcitrol, growth hormones such as thyroid stimulating hormone, cortisol, calcitonin and insulin.

Preferably, hormones for use according to the invention include sex hormones such as progesterones, oestrogens and thyroid hormones such as triiodothyronine (T3) and humic substances (naturally occurring polysaccharides). Particularly preferably the hormone is an oestrogen.

As used herein, ‘an oestrogen’ is any compound which has oestrogenic activity. Included within this definition are naturally occurring or synthetic oestrogen hormones, including phyto-oestrogens, and conjugates, metabolites and derivatives thereof and oestrogen mimetics which may or may not have oestrogen-like molecular structure.

Naturally occurring and synthetic oestrogens are well known in the art and are described for example in ‘Osteoporosis—a clinical guide’ 2^{nd} Edition, Chapter 8, edited by A. D. Wolf and A. St. John Dixon (Martin Dunitz Ltd. 1998).

Suitable naturally occurring oestrogens for use according to the invention include estrone, estropipate, equilin, estradiol, estradiol valerate, ethinyl estradiol, quinestrol and estriol. These may be obtained, for example, from pregnant mares' urine or may be prepared by conventional synthetic methods. Synthetic oestrogens which may be used according to the invention include, for example, dienestrol, mestranol.

Conjugated oestrogen is a mixture of oestrogens, obtained from natural sources, in the form of the sodium salts of the water-soluble sulphate esters of estrone and equilin which may contain smaller amounts of other oestrogenic substances such as 17 alpha dihydroequilin, 17 alpha estradiol, equilenin and 17 alpha dihydroequilenin as salts of their sulphate esters.

Preferred oestrogens for use according to the present invention include conjugated oestrogen and particularly estradiol and its derivatives such as estradiol valerate.

It will be appreciated that the invention is intended to extend to the use of more than one hormone in combination therapy.

Reference herein to ‘an oestrogen’, for example, will therefore be understood accordingly to mean one or more oestrogens unless otherwise indicated.

It will also be appreciated that the one or more hormones, for example one or more oestrogens may advantageously be used in conjunction with one or more other therapeutic agents in addition to the silicon containing component. In oestrogen replacement therapy, for example, it is known to be advantageous to administer a progesterone, such as norethisterone acetate, in combination with an oestrogen in order to reduce the risk of cancer. Contraceptive treatments also typically contain oestrogen in combination with a progesterone.

Other components which may also be included include other therapeutic agents which have been proposed to be effective in conditions which may be prevented or treated by enhancing hormone levels such as oestrogen levels. For example, soluble boron compounds such as boric acid (U.S. Pat. No. 4,849,220), vitamin D and its metabolites and analogues, bisphosphonates and other bone-active phosphonates (see for example, U.S. Pat. No. 6,329,354) and isoflavones (see for example U.S. Pat. No. 6,326,666) and parathyroid hormone (PTH) or PTH-like molecules.

It is to be understood that the present invention also covers the use of a hormone, such as an oestrogen, and a silicon containing component in combination with one or more other therapeutic agents.

A ‘silicon containing component’ for use according to the invention is any silicon containing material which, upon administration to a human or animal subject, is capable of being taken up and utilised by cells, or which, upon administration to a human or animal subject, may be converted to a compound which can be taken up and utilised by cells.

Suitable silicon containing materials for use according to the invention include silicates, silicic acids, silanols and other organo-silicon compounds including reaction products produced by reacting a silicate ester with ascorbic acid or an ascorbic acid derivative, as described for example in U.S. Pat. No. 4,870,191.

According to one preferred embodiment, the silicon containing component for use according to the invention is silicic acid including polysilicic acids, especially orthosilicic acid.

Alternatively, the silicon containing material may suitably be any material which releases silicic acid, especially orthosilicic acid, or polysilicic acids upon administration to a human or animal subject.

According to one embodiment, the silicon containing material may suitably be elemental silicon, for example amorphous silicon or bulk crystalline silicon, preferably polycrystalline silicon and especially porous silicon or silicon particles with a surface area exceeding 25 m²/g. Porous silicon, in particular, is degraded in the body to produce orthosilicic acid.

Porous silicon may be classified depending upon the nature of the porosity (the porosity is the fractional void content by volume). Microporous silicon has an average pore size of less than 2 nm, mesoporous silicon has an average pore size between 2 and 50 nm and macroporous silicon contains pores having a diameter greater than 50 nm. Certain forms of porous silicon are resorbable, that is they dissolve, at normal physiological temperatures (37° C.±1° C.) in simulated body fluid over a period of time, for example of up to 8 weeks and generally at less than 2 weeks.

It will be appreciated that the nature of the porosity of the porous silicon used according to the invention will depend in part on the intended mode of delivery but preferably the porous silicon is microporous or mesoporous, especially mesoporous.

Preferably, the porous silicon for use according to the invention has a porosity of between 1% and 99%, preferably between 20% and 90%, especially between 40% and 80%.

Preferably the porous silicon is resorbable.

The invention relates in particular to HRT and especially for methods for the prevention, treatment and management of osteoporosis. The invention also relates to methods for contraception.

It will be appreciated that reference to treatment includes prophylaxis unless otherwise indicated.

The method according to the invention involves administering hormone in an amount per day which is lower than the usual effective dose of hormone per day in the treatment in the absence of the silicon containing compound. As used herein, the ‘normal effective dose of hormone per day in the treatment’ is the normal dose of hormone alone which is effective in eliciting the desired response

It will be appreciated that the normal effective dose will vary depending on the particular hormone concerned, the treatment involved and the method of administration. In the case of HRT, dosage regimens based on low, medium and high doses of oestrogen are currently in use, as described for example in ‘Osteoporosis-a clinical guide’ 2^{nd} Edition, Chapter 8, referred to above.

Typical daily dosages for conjugated oestrogens in low, medium and high dose osteoporosis therapy are 0.3 mg, 0.625 mg and 1.25 mg respectively. Micronised 17 beta estradiol is typically administered in daily doses of 1 mg (low), 2 mg (medium) and 4 mg (high) but when administered transdermally, the daily doses are suitably 0.025 mg (low), 0.05 mg (medium) and 0.1 mg (high). In the case of estradiol valerate, low dose therapy typically involves administering a dose of 1 mg per day; high dose therapy with the same oestrogen typically involves a total daily dose of 2 mg.

By administering a silicon containing material with an oestrogen according to the present invention, the daily dose of oestrogen required for effective prevention or treatment of osteoporosis is reduced compared to the dose which would be required in the absence of the silicon containing material. In particular, the present inventors have found that not only is the anti-osteoporosis effect of the silicon containing component and oestrogen together greater than when either the oestrogen or silicon containing component are administered alone but the effects are also greater than the sum of the individual effects.

The oestrogen is preferably administered daily to provide a daily dose which is lower than the normal effective dose of oestrogen per day in the treatment in the absence of the silicon containing component.

Preferably, the daily dose of oestrogen according to the invention is from 10% to 90%, especially from 10% to 70%, of the normal effective dose of oestrogen per day in the treatment in the absence of the silicon containing compound.

Preferably the silicon containing component is administered daily to provide, orally, a daily dose of 10-700 mg silicon, more preferably 15-500 mg, ideally 20-100 mg. For non-oral routes, the silicon containing material will be administered daily, preferably to increase fasting, circulating silicon levels by 20% or more.

The oestrogen and the silicon containing components for use according to the present invention may be administered together or sequentially

According to one embodiment, the invention relates to methods for HRT especially treating or preventing osteoporosis in a human or other animal subject by preventing, reducing or reversing bone loss. Particularly preferably, the invention relates to a method for preventing osteoporosis in post-menopausal females.

In methods for preventing osteoporosis according to the invention, treatment with the oestrogen and silicon containing components typically is performed over a period of at least 2 months, preferably greater than or equal to 2 years, ideally for life.

According to another embodiment, the oestrogen and silicon containing components may be administered according to the invention to treat human or other animal subjects with established osteoporosis in order to bring about an increase in bone mass. Typically, this involves administering the oestrogen and silicon containing components over a time period of at least 2 months, preferably greater than or equal to 2 years, ideally for life. Again, it will be appreciated that the time necessary to achieve the desired effect will depend, for example, on inter-individual variation including host factors, other medication being taken and other environmental factors, and will readily be determined according to normal clinical practice.

The hormone and silicon containing components are conveniently administered in the form of pharmaceutical compositions and may conveniently be presented for use in conventional manner in admixture with one or more physiologically acceptable carriers or excipients for administration by any suitable manner conventional in human or veterinary medicine. Suitable methods of administration include, for example, oral, parenteral or topical administration. Alternatively, either or both of the hormone and silicon containing components may be presented in a form suitable for implantation.

For oral administration, the pharmaceutical compositions used according to the invention may take the form of, for example, tablets, capsules, solutions, syrups or suspensions prepared by methods conventional in the art.

For parenteral administration, the compositions may be given as an injection or as an infusion (for example, intravenously, intramuscularly or subcutaneously).

Compositions for topical administration may be formulated, for example, as creams, patches or other such topical formulations conventional in the art.

Preferably, the compositions are administered orally or transdermally.

Physiologically acceptable carriers or excipients suitable for administration to a human or other animal subject according to the invention are well known in the art. It will be appreciated that the choice of carrier or excipient will depend on the intended mode of administration. Suitable carriers or excipients for oral administration include those conventional in the art, for example, sugars, such as lactose, saccharose, glucose; starches, for example potato starch, corn starch; celluloses or derivatives such as sodium carboxymethylcellulose, ethyl cellulose; binders such as gelatine, polyvinylpyrrolidone; lubricants such magnesium stearate, polyethylene glycol, waxes, paraffins and the like.

The hormone and the silicon containing compound for use according to the present invention may be administered at the same time or sequentially. For simultaneous administration the active components may be provided in separate compositions or preferably they may be formulated together in a single composition for co-administration. Suitably the active components may be administered in unit-dose form, preferably orally in the form of tablets, capsules, solutions and the like or transdermally, especially in the form of a patch.

Conveniently, the hormone and silicon containing components may be provided in the form of a kit suitably comprising a hormone, for example an oestrogen, a silicon containing component and instructions, packaging and optionally dispensing means.

The present invention may be illustrated by the following non-limiting examples, when read together with the accompanying figures in which:—

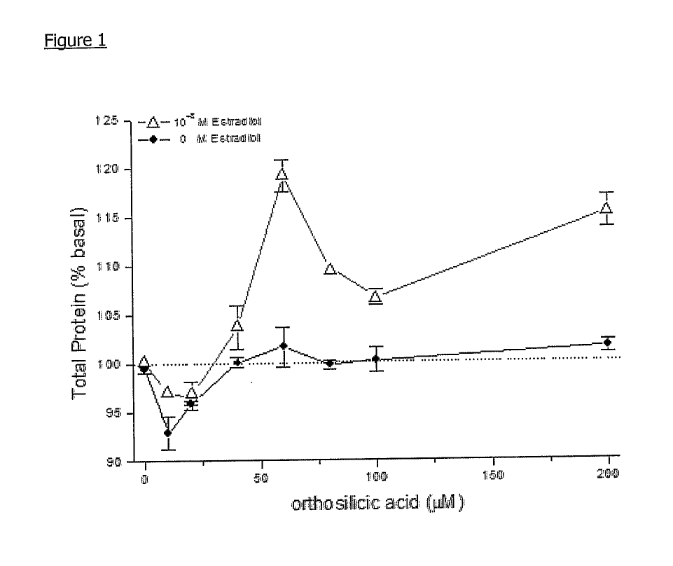

FIG. 1-5 show the effect of various hormones and orthosilicic acid on total protein content for pre-osteoblasts and other osteoblast-like cells.

### EXAMPLE 1

A cell culture model was developed in which osteoblast cells were grown under low oestrogen conditions prior to the start of the test. Osteoblast cells, which are readily available commercially, were then exposed to orthosilicic acid and oestrogen. FIG. 1 shows the effect of orthosilic acid on total cell protein, and the influence of estradiol upon this behaviour.

Co-incubation of osteoblast cells with the oestrogen and orthosilicic acid was found to increase total cell protein, relative to the total protein observed when orthosilicic acid was used in the absence of oestrogen; a similar increase in type I collagen production was observed for these experimental conditions. The type I collagen and total protein, for the combined treatment, was also higher than that observed for treatment with oestrogen in the absence of orthosilicic acid.

These results show that silicon is required for the biological activity of oestrogen, and that silicon can be used to reduce the dose of oestrogen required for effective oestrogen treatment.

### EXAMPLE 2

Synergistic activity of silicon and hormones on osteoblast protein content.

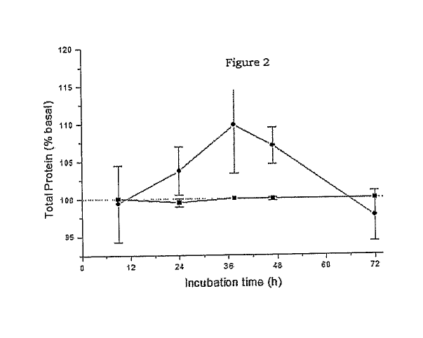

Pre-osteoblast cells, available commercially, were grown in an environment free of oestrogen and oestrogen mimetics and then stimulated with oestradiol (E2), at 10⁻⁹ M, for varying lengths of time in the presence or absence of silicon (orthosilicic acid, 50 micromolar) and total protein concentration was assessed. Clearly, in the absence of silicon, oestradiol had no effect on total protein concentration at any time point versus baseline (cells under identical conditions but with only vehicle, without E2, added) (FIG. 2). Similarly, when cells in an oestrogen-free environment were challenged with orthosilicic acid without E2, there was no significant effect on total protein. However, when cells were challenged with E2 under identical conditions to those above, except that 50 micromolar silicon (Si) was present, there was a clear, time-dependent response in terms of an increase in total protein concentration (FIG. 2). The cells for these respective experiments had also been passaged and maintained in media containing no added silicon (for the subsequent E2 or vehicle alone experiments) or containing 50 micromolar silicon (for the subsequent Si or E2+Si experiments).

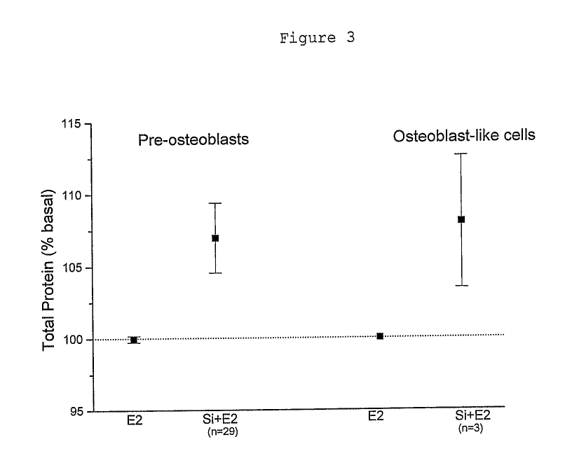

This response was not restricted to pre-osteoblast cells as, in experiments identical to those described above but using just the 48 hour time point, silicon-E2 synergy was similarly observed in osteoblast-like cells of an osteosarcoma cell line (FIG. 3) and also in other osteoblast-like cells (data not shown) indicating that there is broad responsiveness amongst cells of the osteoblast lineage.

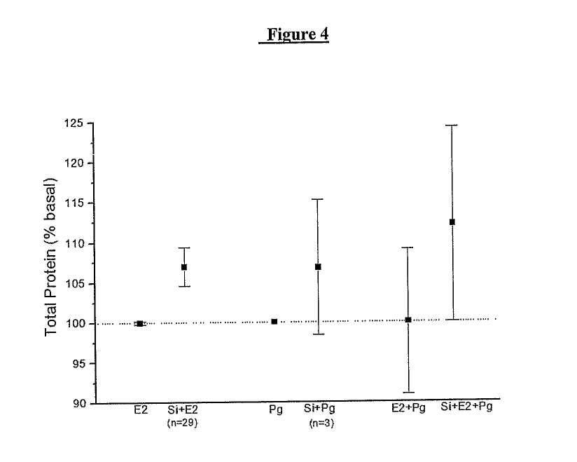

Thus in experiments identical to those described above at the 48 hour time point, we investigated the effects of 10⁻⁶ M progesterone instead of oestradiol. This showed a modest effect together with silicon but not in the absence of silicon (FIG. 4). In a further experiment, there was some evidence for a greater mean effect of progesterone plus oestradiol with silicon versus that of either progesterone alone with silicon or oestradiol alone with silicon (FIG. 4). These results show that the silicon-oestradiol synergy is not lost when progesterone is also added into the system and, secondly, silicon may have some synergistic effect with hormones other than oestradiol.

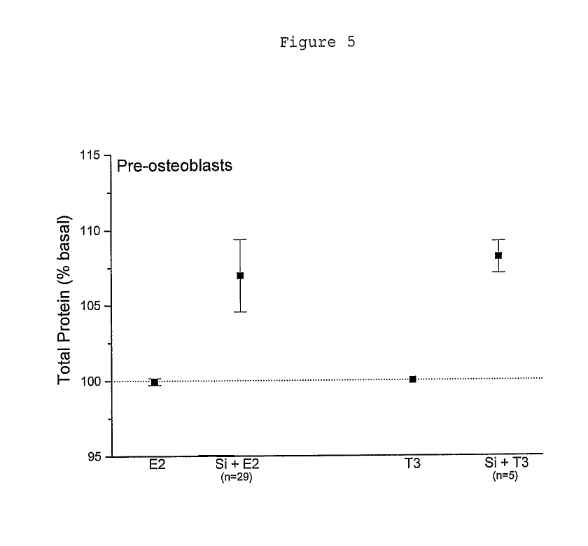

This was confirmed by experiments with triiodothyronine (T3) at 10⁻⁹ M, under experimental conditions otherwise identical to those above for 48 hours incubation (FIG. 5). T3 is a thyroid hormone, with some oestradiol agonist and antagonist properties, indicating that the synergistic effect of silicon may extend to a range of hormones and not be restricted to the sex-hormones or to activity on bone

In other experiments it was confirmed that when substances which are capable of stimulating osteoblasts but which are not hormones are administered, this synergistic silicon effect is not observed.

It will be obvious to those skilled in the art that an increase in total protein in cells is associated with either an increase in cell numbers or an increase in specific proteins or a combination of the two and that this should be beneficial for the functioning of these cells

### EXAMPLE 3

Loading of beta-estradiol and progesterone onto porous silicon

Hormones were loaded onto porous silicon wafers according to the following method:—

A 1 cm² piece of p+ porous silicon (70% porosity, 66 μm) was weighed and then placed on a hot plate and heated to about 150° C. A small amount of progesterone powder was placed on the porous silicon wafer surface, evenly distributed over the wafer surface. Once the powder had melted, the wafer was kept on the hot plate for another 5 minutes, then cooled to room temperature. The wafer surface was washed with ethanol a few times, dried and then weighed again.

Loading of β-Estradiol:

The same procedure was employed as for progesterone, except that the loading temperature for β-Estradiol was 200° C. Both p+ porous silicon wafer and membrane (63% porosity, 150 μm) were used as the loading matrix.

Loading Results

The weights of porous silicon (pSi) samples before and after loading are shown in Table 1. As can be seen from table 1 that 4.02 mg Progesterone was loaded onto the wafer sample, 4.26 mg β-Estradiol was loaded onto the wafer sample. 12.99 mg β-Estradiol was loaded onto the membrane. This is in proportion with the surface area of each sample. The two wafer samples are similar size (˜1 cm²), while the membrane is in irregular shape, its size is around 3 cm².

TABLE 1
Loading Results for Progesterone and β-Estradiol

| | Weight | Weight | Loaded |
|---|---|---|---|
| | before | after | Amount |
| Sample | loading (mg) | loading (mg) | (mg) |
| Progesterone/pSi | 81.14 | 85.16 | 4.02 |
| wafer | | | |
| β-Estradiol/pSi | 86.75 | 91.01 | 4.26 |
| wafer | | | |
| β-Estradiol/pSi | 51.42 | 64.41 | 12.99 |
| membrane | | | |
| Control pSi | 76.66 | 76.96 | — |
| wafer | | | |

### EXAMPLE 4

The following are examples of formulations of oestrogen(s) and a silicon containing component for use according to the invention.

(a) HRT/Osteoporosis Patch

Circular silicon discs of diameter 5-25 mm and thickness 350-650 microns are sawn out of silicon wafers and isotropically etched to smooth their edges. One face of the discs is rendered mesoporous by anodisation to a thickness of 10-200 microns. A blend of micronized estradiol and progesterone is introduced into the pores of each disc by contact with the disc at a temperature in the range 176° C. to 200° C. for between 5 minutes and 60 minutes. A protective sealing layer, whose diameter exceeds that of the silicon disc, is applied to the front face of the disc. An adhesive backing layer whose diameter also exceeds that of the silicon disc, is mechanically applied to the rear face. Immediately prior to application to the skin, the front sealing layer is removed.

(b) Oral Formulation

Mesoporous silicon membranes of 64% porosity and 150 microns thickness are prepared by anodisation and loaded with beta-estradiol by placing the estradiol on the sample of porous silicon and heating it until it melts and passes into the pores of the porous silicon. Similar membranes are loaded with progesterone by the same technique. The membranes are then mechanically crushed in a ball miller and classified to achieve microparticle size distributions in the range 10-150 microns diameter. Microparticles loaded with one hormone are then mixed with those of the other in the appropriate ratio. The drug loaded microparticles are then mechanically compressed into tablets. An orally acceptable binding agent may be used. An orally acceptable excipient coating is then applied to the tablets, to make them more palatable.

(c) Alternative Oral Formulation

Polycrystalline silicon granules are jet milled and classified to have a defined size distribution in the range 10-150 microns diameter. These are then stain etched using conventional methods to give a porous coating. The particles are then co-milled with micronised oestrogen particles to achieve at least partial impregnation of the mesoporsity. Excess oestrogen residing on the outer microparticle surface is reduced by washing with a suitable solvent that does not wet porous silicon efficiently. The drug loaded microparticles are then mechanically compressed into tablets. An orally acceptable binding agent may be used. An orally acceptable excipient coating is then applied to the tablets, to make them more palatable.

1. Use of a silicon containing component to reduce the amount of hormone required to be administered per day to a human or animal subject in order to achieve the desired therapeutic effect in a method of hormone treatment.

2. Use according to claim 1 wherein the hormone has oestrogen pathway interacting activity or bone influencing activity.

3. Use according to claim 1 wherein the hormone is a sex hormone or a thyroid hormone.

4. Use according to claim 1 wherein the hormone has oestrogenic activity.

5. Use according to claim 1 in hormone replacement therapy

6. Use according to claim 1 in treating or preventing osteoporosis.

7. Use according to claim 1 wherein the silicon containing component is selected from silicic acid, porous silicon or silicon particles with a surface area exceeding 25 m²/g.

8. Use of a hormone and a silicon containing component in the manufacture of a medicament for the treatment or prevention of a condition in a human or other animal subject in which enhancing the level of hormone is indicated to be beneficial, wherein the amount of hormone administered per day is lower than the normal effective dose of hormone per day in the treatment in the absence of the silicon containing component.

9. Use according to claim 8 wherein the hormone is an oestrogen hormone.

10. Use according to claim 9 in the manufacture of a medicament for the treatment or prevention of osteoporosis.

11. Use according to claim 9 wherein the oestrogen is selected from estrone, estropipate, equilin, estradiol, estradiol valerate, ethinyl estradiol, quinestrol, estriol, dienestrol, mestranol, conjugated oestrogen or a mixture thereof.

12. Use according to claim 8 wherein the silicon containing component is selected from silicic acid, porous silicon or silicon particles with a surface area exceeding 25 m²/g.

13. Use according to claim 9 wherein the oestrogen is administered in an amount of from 10% to 90% of the normal effective dose of oestrogen per day in the treatment in the absence of the silicon containing component.

14. Use according to claim 9 wherein the oestrogen is administered in an amount of from 10% to 70% of the normal effective dose of oestrogen per day in the treatment in the absence of the silicon containing component.

15. Method for treating or preventing a condition in which enhancing the level of hormone is indicated to be beneficial in a human or other animal subject comprising administering to the subject a silicon containing component and administering to the subject a hormone in an amount per day which is lower than the normal effective dose of hormone per day in the treatment in the absence of the silicon containing component.

16. Method according to claim 15 wherein the hormone has oestrogenic activity.

17. Method according to claim 15 for treating or preventing osteoporosis.

18. Method according to claim 15 wherein the hormone is selected from estrone, estropipate, equilin, estradiol, estradiol valerate, ethinyl estradiol, quinestrol, estriol, dienestrol, mestranol, conjugated oestrogen or a mixture thereof.

19. Method according to claim 15 wherein the silicon containing component is selected from silicic acid, porous silicon or silicon particles with a surface area exceeding 25 m²/g.

20. Method according to claim 16 wherein the oestrogen is administered in an amount of from 10% to 90% of the normal effective dose of oestrogen per day in the treatment in the absence of the silicon containing component.

21. Method according to claim 20 wherein the oestrogen is administered in an amount of from 10% to 70% of the normal effective dose of oestrogen per day in the treatment in the absence of the silicon containing component.

22. A pharmaceutical composition for co-administration comprising a silicon containing component and an oestrogen, wherein the oestrogen is present in an amount which is lower than the normal effective dose of oestrogen per day in the treatment in the absence of the silicon containing component.

23. Use of porous silicon and oestrogen in the manufacture of a medicament for the treatment or prevention of a condition in which enhancing the level of oestrogen is indicated to be beneficial in a human or other animal subject.

24. Method of treating or preventing a condition in which enhancing the level of oestrogen is indicated to be beneficial in a human or other animal subject comprising administering to the subject porous silicon and an oestrogen.

25. A pharmaceutical composition comprising porous silicon and an oestrogen.

26. Use of a composition according to claim 25 in therapy in a human or other animal subject.
